(19) United States
(12) Patent Application Publication
(10) Pub. No.: US 2006/0229518 A1
(43) Pub. Date: Oct. 12, 2006

(54) Method for continuous, non-invasive, non-radiating detection of fetal heart arrhythmia

(75) Inventor: Eran Ofek, Modiin (IL)

Correspondence Address: DR. MARK FRIEDMAN LTD.;c/o Bill Polkinghorn, 9003 Florin Way, Upper Marlboro, MD, 20772, US

(73) Assignee: PHYSICAL LOGIC AG

(21) Appl. No.: 11/094,262

(22) Filed: Mar. 31, 2005

(51) Int. Cl. A61B 5/02

(52) U.S. Cl. 600/500; 607/902; 600/508

(57) ABSTRACT

A method to analyze the fetus's heartbeat signal and detect a specific list of FHR arrhythmias in a non-intrusive, non-invasive and non-emitting way. The method comprises passively sensing a plurality of micro-vibration signals transmitted through the mother's body; and extracting at least one fetal heart arrhythmia parameter from said micro-vibration signals, using maternal characteristics as an added input.

### FIELD OF THE INVENTION

The present invention relates in general to fetal monitoring, and in particular to the automatic detection and analysis of fetal cardiac arrhythmias using body micro-vibrations.

### BACKGROUND OF THE INVENTION

The position of a fetus within the womb, where it is surrounded by amniotic fluid, makes examination of the fetus very difficult for most examination techniques. Existing methods for monitoring, detecting and analyzing fetal heart rate problems have problems long acknowledged by the medical profession.

Traditional methods for measuring the fetal heart rate use ultrasound devices and various fetal monitors. All such methods are limited by the way the information is obtained and by the process of analyzing the results, and suffer from a number of common problems:

Intrusive—both an ultrasound and a fetal monitor require the mother to come to the testing facility and either have a monitor strapped on her abdomen (Fetal Monitor), or a conductive gel applied to the abdomen, followed by the use of a transducer (Ultrasound). Both solutions require special effort on part of the mother and use of major medical equipment.

Radiative—both ultrasound and fetal monitors transmit some sort of signal in order to obtain information about the fetus. In ultrasound, this is an ultrasonic pulse, transmitted directly toward the fetus through the conductive gel, whereas in a fetal monitor (such as the one used in a delivery room) the principle is much the same. In the sense that both solutions require transmission of a signal directly toward the fetus, the methods are “radiative”.

Invasive—for example, the fetal monitor in a delivery room requires an electrode to be an inserted into the mother to touch the head of the fetus.

The heart of the mother and the fetus are both organs with a mass of matter that moves and pushes fluids within their respective bodies, thereby generating internal vibrations within liquid media. The combined mother and fetus masses are represented by a body that has a “combined” center of gravity. The combined center of gravity moves in response to the movement caused by the pushing of fluids by the respective hearts to compensate accordingly, resulting in micro-vibrations that propagate through the mother's body. These micro-vibrations can advantageously be detected by a vibration sensor attached to the body.

Detection of body micro-vibration is known in the art, see for example R. Strum, R, B. Nigg and E. A. Koller, “The impact of cardiac activity on triaxially recorded endogenous micro-vibrations of the body”, European Journal of Applied Physiology, vol. 44, pp. 83-96, 1980. Strum et al. evaluated the relationship between the cardiac activity and the micro-vibrations of the body and concluded that the most important source of whole-body micro vibrations is the cardiac activity.

Active micro-vibration fetal heart monitoring is known, as disclosed for example in U.S. Pat. No. 6,454,716 and US Patent Applications Nos. 20030153831 and 20030153832 to Zumeris. The methods disclosed therein require an active element to generate vibrations or send a signal through the mother's body in order to detect parameters related to the fetal heartbeat.

Passive fetal monitoring that involves sensing of body vibrations is also known, as disclosed for example in U.S. Pat. No. 6,135,969 to Hale et al., US Patent Applications Nos. 20020068874 to Zuckerwar et al and 20030120159 to Mohler, and the references cited therein. Hale discloses a vibration sensor comprising a circular layer of piezoelectric material supported on a larger substrate disc of a thin, somewhat flexible material. Flexing of the disc generates an electrical signal. The substrate disc is weighted at its periphery, and the central portion of the disc is supported on a member for transferring motion or vibration that it is desired to detect. In the preferred embodiment the center support is provided by a pair of bosses projecting into the otherwise hollow interior of a two-part casing that encloses the disc-weight assembly. The weights impart a peripheral inertia that makes the composite unit sensitive to minute vibrations. Hale et al. does not indicate how the sensor may be used for FHR monitoring. Zuckerwar discloses a fetal heart monitoring system and method for detecting and processing acoustic fetal heart signals transmitted by different signal transmission modes. One signal transmission mode, the direct-contact mode, occurs in a first frequency band when the fetus is in direct contact with the maternal abdominal wall. Another signal transmission mode, the fluid propagation mode, occurs in a second frequency band when the fetus is in a recessed position with no direct contact with the maternal abdominal wall. The second frequency band is relatively higher than the first frequency band. The fetal heart monitoring system and method detect and process acoustic fetal heart signals that are in the first frequency band and in the second frequency band. Zuckerwar's system and method do not refer to directly, and are not concerned even indirectly with fetal heart induced micro-vibrations and their measurement. Mohler discloses an apparatus, operation and method for measurement of systemic and/or pulmonic blood pressure. The non-invasive measurement is done through detection, identification and characterization of the second heart sound acoustic signature associated with heart valve closure, through an acoustic sensor. Thus Mohler's system and method also do not refer to directly, and are not concerned even indirectly with fetal heart induced micro-vibrations and their measurement.

The mother's characteristics, in particular race, are known to affect the signals in fetal monitoring. For example, Johnson et al. in the American Journal of Obstetrics and Gynecology, vol. 17, pp. 779-783, 1998 have shown (Table II) that fetuses of black women have a much higher percent of beat-to-beat variability, variable decelerations and late decelerations than those of women of other ethnic origins. Yeo et al. in the Journal of Maternal-Fetal Investigation, vol. 16, pp. 163-167, 1996 have concluded that the mother's ethnic origin affects the FHR base line. However, none of the vibration-based fetal monitoring methods (either passive or active) take into account the mother's characteristics.

There is thus a widely recognized need for, and it would be highly advantageous to have, a micro-vibration based method and apparatus for fetal monitoring that takes into account the mother's characteristics and which provides continuous and automatic information about the fetal heart condition in a non-intrusive, non-invasive and non-radiating manner.

### SUMMARY OF THE INVENTION

The present invention discloses a method and apparatus to automatically detect and analyze parameters related to the state of a fetal heart, specifically the existence of specific arrhythmias, in a non-invasive, non-intrusive and a non-radiating way, while accounting for maternal characteristics. The method is based on continuous monitoring of micro- or nano-vibrations (hereinafter “micro-vibrations”) that are transferred through the mother's body. These micro-vibrations are correlated with the fetal heart rate (FHR) and fetal movements, and relevant information regarding various fetal heart parameters is extracted from them using digital signal processing algorithms. The parameters may include FHR Base Line, FHR Acceleration, FHR Reactivity, FHR Silent Pattern, FHR Mild Deceleration, FHR Prolonged Deceleration, FHR Bradycardia, FHR Baseline Bradycardia, and FHR Baseline Tachycardia. While analyzing the fetal heartbeat pattern the present invention takes into account maternal characteristics such as the mother's race and the behavior of the maternal heartbeat signal.

The heart operation is cyclic. Both the mother and the fetus have a cyclic micro-movement frequency, which is almost constant over one heartbeat cycle. By using a plurality of micro-vibration sensors located at different positions relative to mother and fetus, we can relationally subtract the noises and filter out the required desirable signals. A sensor that is located closer to the mother's own heart would sense her heart in a stronger signal than a sensor that is located closer to the fetal heart. Although all sensors would sense the mother's heartbeat, by differentiating the signals received from other sensors, we identify the signals that are generated by the fetus. This is done by implementing commonly known mathematical algorithms for signal processing within the DSP element (as part of the signal filtering process).

According to the present invention there is provided a method for automatically detecting and analyzing heartbeat related arrhythmias of a fetus carried by a mother in a non-invasive, non-intrusive, non-radiating way comprising steps of passively sensing a plurality of micro-vibration signals transmitted through the mother's body, and extracting at least one fetal heart arrhythmia parameter from the micro-vibration signals, using maternal characteristics as an added input.

According to one aspect of the method of the present invention, the step of extracting includes extracting a parameter selected from the group consisting of a FHR (Fetal Heart Rate) Base Line parameter, a FHR Acceleration parameter, a FHR Silent Pattern parameter, a FHR Mild Deceleration parameter, a FHR Prolonged Deceleration parameter, a FHR Bradycardia parameter, FHR Baseline Bradycardia parameter and a FHR Baseline Tachycardia parameter.

According to another aspect of the method of the present invention, the step of sensing includes sensing the micro-vibration signals using at least two micro-vibration sensors disposed proximal to the mother's body.

According to yet another aspect of the method of the present invention, the sensing using at least two micro-vibration sensors includes sensing a signal that exhibits a difference in a micro-vibration parameter in each sensor, the micro-vibration parameter selected from the group consisting of a micro-vibration time lag, a micro-vibration amplitude and a combination thereof.

According to yet another aspect of the method of the present invention, the step of extracting includes obtaining a filtered fetal heart rate using the micro-vibration signals, and using the filtered fetal heart rate and the maternal characteristics as inputs to an extraction algorithm which outputs the at least one fetal heart arrhythmia parameter.

According to yet another aspect of the method of the present invention, the usage of the maternal characteristics includes using maternal race.

According to the present invention there is provided a method for automatically detecting and analyzing heartbeat related arrhythmias of a fetus carried by a mother, comprising the steps of passively sensing a plurality of micro-vibration signals transmitted through the mother's body; extracting fetal heart rate signals from the micro-vibration signals, providing maternal characteristics, and combining the fetal heart rate signals and the maternal characteristics to extract at least one fetal heart arrhythmia parameter.

According to one aspect of the method of the present invention, the step of passively sensing includes sensing the micro-vibration signals through at least two sensors, operative to receive each signal with a parameter difference.

According to another aspect of the method of the present invention, the parameter difference is selected from the group consisting of a time lag, a micro-vibration signal amplitude and a combination thereof.

According to another aspect of the method of the present invention, the step of providing maternal characteristics includes providing the maternal race.

### BRIEF DESCRIPTION OF THE DRAWINGS

Exemplary non-limiting embodiments of the invention are described in the following description, read with reference to the figures attached hereto. Dimensions of components and features shown in the figures are chosen primarily for convenience and clarity of presentation and are not necessarily to scale. The attached figures are:

FIG. 1 shows a flowchart of the main steps of the method of the present invention;

FIG. 2 represents a read-out of a fetal heart rate (FHR) signal with specific information;

FIG. 3 is an exemplary calculated beat spectrograph used to analyze and detect arrhythmias according to the present invention;

FIG. 4 is a representation of the input signal from the micro-vibrations sensors, showing example of a combined signal, an MHR signal and a FHR signal;

FIG. 5 is a representation of the filtering process, in which the noise and MHR are subtracted from the input signal to provide the filtered fetal heart rate.

### DETAILED DESCRIPTION OF THE PREFERRED EMBODIMENTS

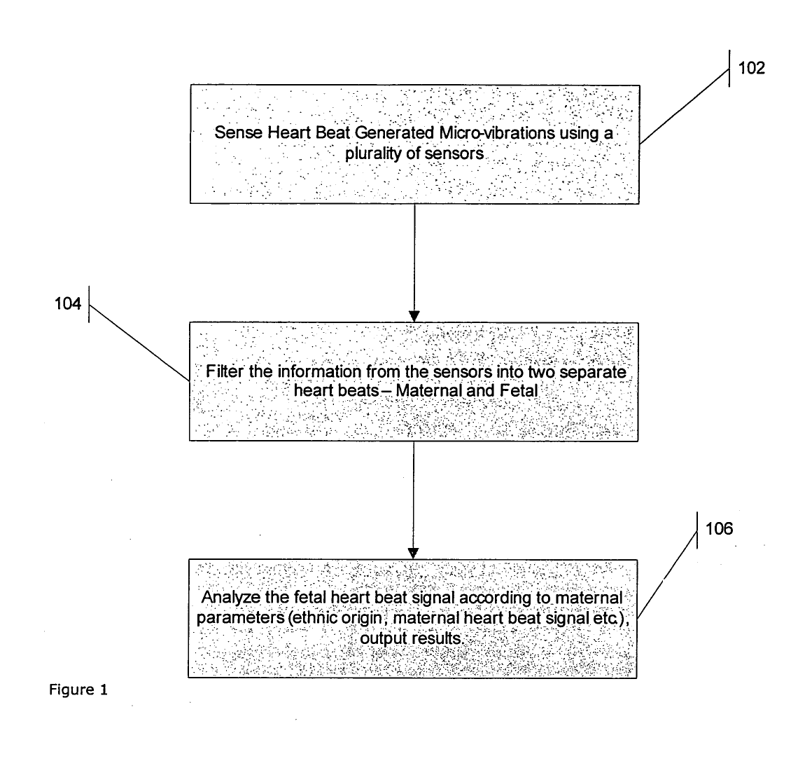

FIG. 1 shows a schematic flow chart describing the main steps of the method of the present invention. In step 102, analog micro-vibration signals are detected using at least one, and more preferably a plurality of sensors located in desired positions around the mother's body, preferably around her abdominal area. “Micro-vibration” in the present disclosure includes “nano-vibration” i.e. vibration processes on both nanometer and micrometer scale. The micro-vibration sensors sense the combined micro-vibration signals produced by the heart motions of both mother and fetus. Micro-vibration sensors applicable for the purposes of the present invention include for example the micro vibrations sensor described in U.S. Pat. No. 6,621,278. The micro-vibration signals are first converted from analog to digital so that they can be processed by a processing element in step 104, for example a digital signal processing (DSP) element. The DSP element performs a signal filtration and noise reduction from the input signal as well as subtracts the mother's heartbeat signal from the information received by the sensors. The removal is based on the fact that the mother's heart rate cyclic frequency is constant and differs from the frequency of the fetus's heart rate. By applying conventional algorithms known in the art, one can subtract the mother's heartbeat signal and receive the fetus's heartbeat signal. An exemplary procedure for filtering maternal and fetal heartbeat signals may be found in U.S. Pat. No. 6,751,498. The filtered maternal and fetal heartbeat signals are then saved into a memory element. In step 106, the information stored in the memory element is analyzed, and required heart parameters are extracted using calculations known in the art, for example as described by Van-Leeuwan et al. in BMC Physiology, volume 3, 2003, and Yeo et al., Journal of Maternal-Fetal Investigation, vol 16 (1996) pages 163-167. These calculations include calculating the base line (which is the common value in a 10 minute measurement) and detecting arrhythmias, which are variations from the base line on a time axis of between 20 and 40 minutes. Inventively, and in contrast with all prior art methods for extraction of fetal arrhythmia parameters, the present invention uses the maternal characteristics as an integral input in the extraction step. As mentioned, Yeo et al. have concluded that the mother's ethnic origin affects the FHR base line. By using the maternal ethnic origin as an added input, the base line value and any other measurements take into account the statistical information relating to the effects of the maternal ethnic origins on the FHR. Further inventively and in contrast with all prior art, the maternal characteristics so used are included in signals obtained with micro-vibration measurements. The extracted parameters may include FHR Base Line, FHR Acceleration, FHR Reactivity, FHR Silent Pattern, FHR Mild Deceleration, FHR Prolonged Deceleration, FHR Bradycardia, FHR Baseline Bradycardia, and FHR Baseline Tachycardia.

All the micro-vibration sensors located around the mother's body receive the same information, but with a difference of time and amplitude. For example, the signal sensed by at least one sensor (e.g. a sensor A) located in the mother abdomen area will differ from the same signal sensed by at least one other sensor (e.g. a sensor B) located near the mother's heart area in either time lag, amplitude or both. Exemplarily, a vibration sensed as “strong” by sensor A, and weaker and delayed by sensor B has probably originated near sensor A. If the “abdomen” sensor detects a heartbeat, and the same heartbeat is detected at a “chest” sensor, but with a delay and with lesser amplitude, this heartbeat is probably a fetal heartbeat. Conversely, if the heartbeat is detected (through the micro-vibration) first by the “chest” sensor, and detected later and weaker by the abdomen sensor, this heartbeat may be confidently attributed to the mother's heart. Noises that are detected by all sensors more or less at the same amplitude and time are considered to be external noises, not related to the heartbeats of the mother and fetus. Thus the method of the present invention can differentiate through micro-vibration measurements between the fetal heartbeat and the maternal heartbeat, and use signal filtering and processing to obtain the fetal arrhythmia parameters, while taking into consideration the maternal characteristics.

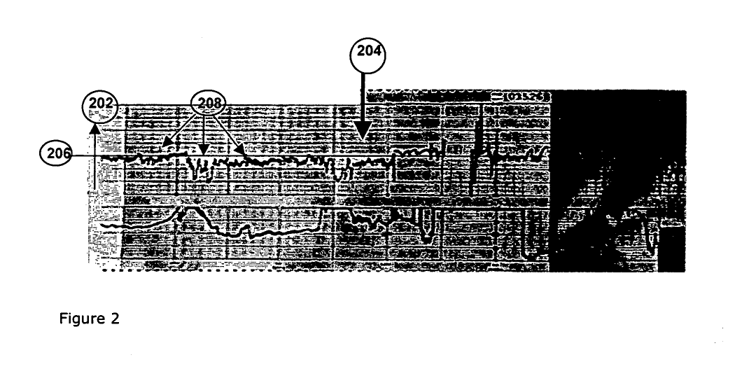

FIG. 2 displays an output of a fetal heart rate signal, where the Y Axis 202 represents the fetal heartbeat rate (FHR). A normal value for FHR is 120-160 beats per minute (BPM). A change 204 of up to 20 beats for a duration of up to 1 minute is also considered normal. The best known parameter upon which all arrhythmia detection algorithms depend upon is the “FHR base line” 206, which is the most common FHR value during the previous 10 minutes. A minimum change 208 of FHR values from the last FHR base line values of up to 5 BPM should appear during 20% of the sampling time (see Williams Obstetrics, 21st edition; 2001 pages 334-337).

All arrhythmias are considered as FHR values variations from the base line over a defined duration of time. In order to detect the existence of arrhythmias, the sampling time frame should be between 20 and 40 minutes. The disclosed method simply follows the known rules of the art in order to detect and identify FHR related arrhythmias. However, in contrast with normal procedure, the maternal characteristics are used as an integral part (input) in this identification.

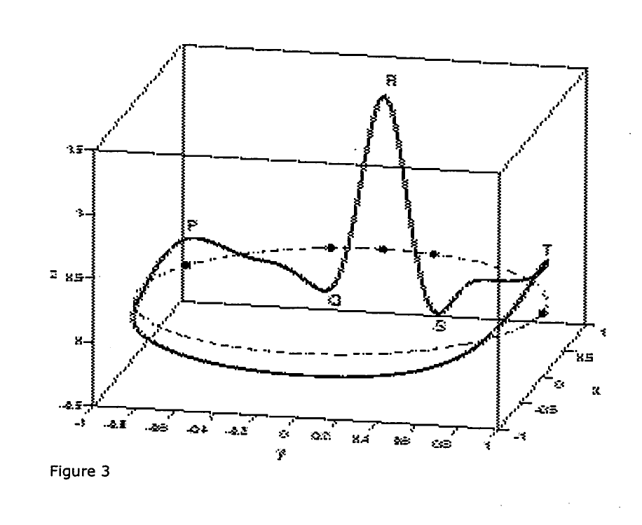

FIG. 3 displays a calculated beat spectrograph according to which known phenomena and syndromes are analyzed. The figure shows a three dimensional graph which represents the operational cycle of the heart (of the fetus), where the X, Y and Z axes represent the changes in the center of gravity, while the Z axis is also shifted in order to present those cycles over time and to allow counting and processing of intervening geometrical changes. Shifting the Z axis by time creates a geometrical “spiral like” graph, which represents changes in the center of gravity of the mother heart, fetus heart and other internal/external mass movements.

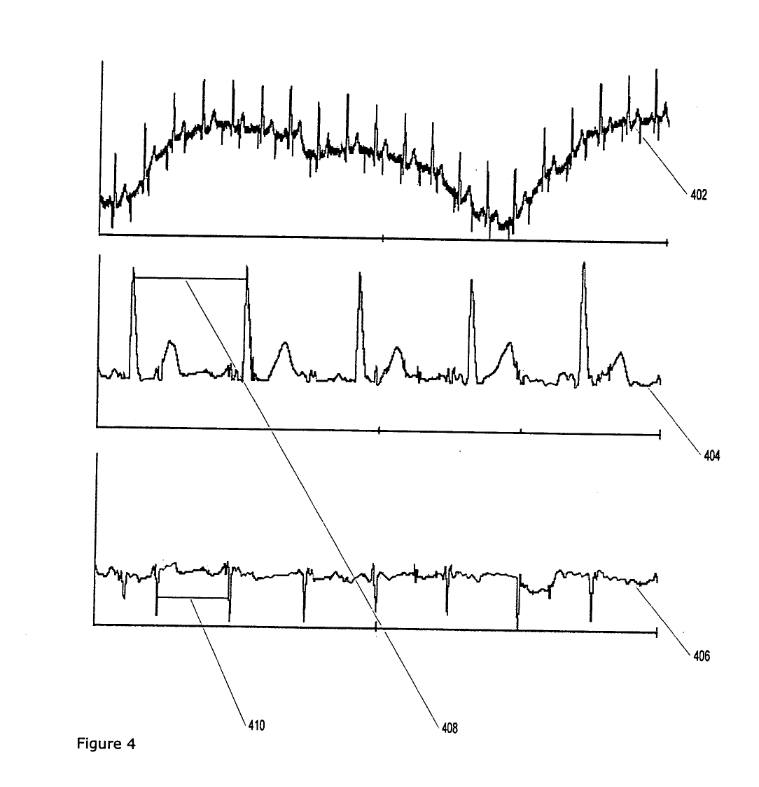

FIG. 4 shows a typical graph 402 (top) containing information received from one or more sensors. The information within graph 402 contains both the maternal and fetal heartbeat signals and includes additional noise. The mother heartbeat rate (MHR) signals 404 (middle graph) and FHR signals 406 (bottom graph) are derived from the top graph. MHR signals 404 have a frequency 408 and FHR signals 406 have a frequency 410. When sensed with micro-vibration sensors, each peak in signals 406 may appear differently at different sensors. In particular, each peak may have a different shape (distortion) and amplitude, and the frequency 410 may vary also between the sensors. A time lag between identical peaks received at different sensors, depending on their location proximal to the mother's body, may also be measured. All these may be used as inputs to the extraction algorithms, in combination with the maternal characteristics.

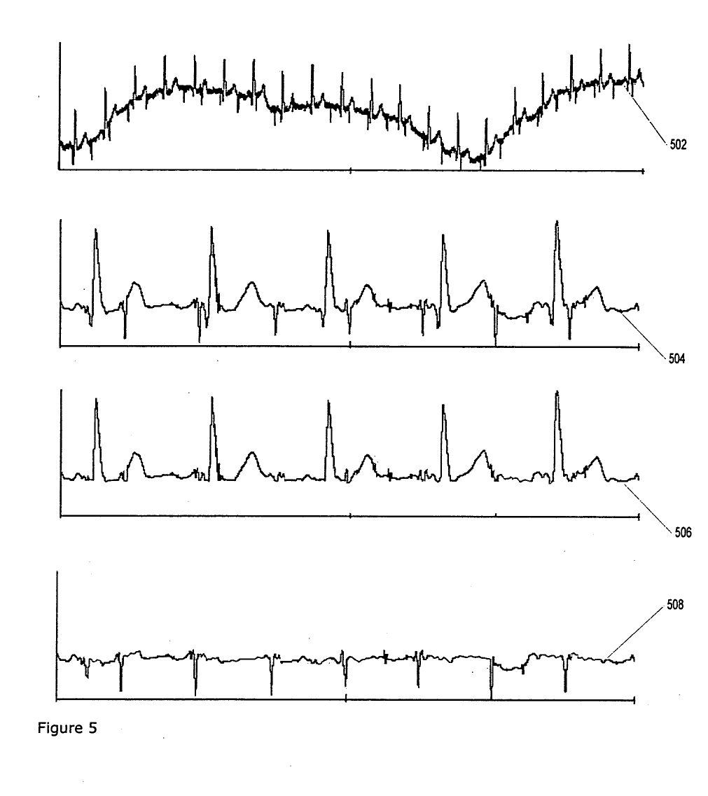

FIG. 5 illustrates an example of the filtering process, where an input signal 502 is the combined signal of mother and fetal micro-vibrations and additional noise (similar to 402 in FIG. 4). The noise is removed, leaving only combined maternal and fetal heart signals 504. The maternal and fetal waveforms are separated (signals 506 and 508 respectively) from the combined signal. The information contained in waveforms 508, is combined with the input of maternal characteristics to advantageously provide the various fetal arrhythmia parameters.

All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

What is claimed is:

1. A method for automatically detecting and analyzing heartbeat related arrhythmias of a fetus carried by a mother in a non-invasive, non-intrusive, non-radiating way, comprising steps of:
a. passively sensing a plurality of micro-vibration signals transmitted through the mother's body; and
b. extracting at least one fetal heart arrhythmia parameter from said micro-vibration signals, using maternal characteristics as an added input.

2. The method of claim 1 wherein said step of extracting includes extracting a parameter selected from the group consisting of a FHR (Fetal Heart Rate) Base Line parameter, a FHR Acceleration parameter, a FHR Silent Pattern parameter, a FHR Mild Deceleration parameter, a FHR Prolonged Deceleration parameter, a FHR Bradycardia parameter, FHR Baseline Bradycardia parameter and a FHR Baseline Tachycardia parameter.

3. The method of claim 1, wherein said step of sensing includes sensing said micro-vibration signals using at least two micro-vibration sensors disposed proximal to the mother's body.

4. The method of claim 3, wherein said sensing using at least two micro-vibration sensors includes sensing a signal that exhibits a difference in a micro-vibration parameter in each said sensor, said micro-vibration parameter selected from the group consisting of a micro-vibration time lag, a micro-vibration amplitude and a combination thereof.

5. The method of claim 1, wherein said step of extracting includes obtaining a filtered fetal heart rate using said micro-vibration signals, and using said filtered fetal heart rate and said maternal characteristics as inputs to an extraction algorithm which outputs said at least one fetal heart arrhythmia parameter.

6. The method of claim 1, wherein said using said maternal characteristics includes using maternal race.

7. A method for automatically detecting and analyzing heartbeat related arrhythmias of a fetus carried by a mother, comprising the steps of:
a. passively sensing a plurality of micro-vibration signals transmitted through the mother's body;
b. extracting fetal heart rate signals from said micro-vibration signals;
c. providing maternal characteristics; and
d. combining said fetal heart rate signals and said maternal characteristics to extract at least one fetal heart arrhythmia parameter.

8. The method of claim 7, wherein said step of passively sensing includes sensing said micro-vibration signals through at least two sensors, operative to receive each said signal with a parameter difference.

9. The method of claim 8, wherein said parameter difference is selected from the group consisting of a time lag, a micro-vibration signal amplitude and a combination thereof.

10. The method of claim 7, wherein said step of providing maternal characteristics includes providing maternal race.

11. The method of claim 7, wherein said step of combining includes combining said fetal heart rate signals and said maternal characteristics to extract at least one parameter selected from the group consisting of a FHR (Fetal Heart Rate) Base Line parameter, a FHR Acceleration parameter, a FHR Silent Pattern parameter, a FHR Mild Deceleration parameter, a FHR Prolonged Deceleration parameter, a FHR Bradycardia parameter, FHR Baseline Bradycardia parameter and a FHR Baseline Tachycardia parameter.
